(19) United States
(12) Patent Application Publication
(10) Pub. No.: US 2005/0053564 A1
(43) Pub. Date: Mar. 10, 2005

(54) Use a plant extract to enhance hair growth and hair restoration for damaged hair

(76) Inventor: Chaim Lieberman, Brooklyn, NY (US)

Correspondence Address: Dr. O. M. (Sam) Zaghmout;Bio Intellectual Property Services LLC, 8509 Kernon CT, Lorton, VA, 22079, US

(21) Appl. No.: 10/934,700

(22) Filed: Sep. 7, 2004

Related U.S. Application Data
(60) Provisional application No. 60/499,919, filed on Sep. 4, 2003.

(51) Int. Cl. A61K 7/06; A61K 7/11; A61K 35/78

(52) U.S. Cl. 424/70.13; 424/74; 424/777

(57) ABSTRACT

The present invention includes methods for the treatment and/or prevention of hair loss and methods for the regeneration or restoration of hair growth comprising a step of identifying an individual suffering from or susceptible to hair loss or hair thinning or in need of hair regeneration, and a step of administering of a plant extract identified as Shea Butter in combination with papaya and polysaccharides. Preferably, the extract is an aqueous extract and is administered topically. The present invention also provides a composition, preferably in the from of a lotion, gel, cream, or other suspension, and a distinct chemical compound or class of chemical compounds therein, effective in restoring hair growth, preventing hair loss, and/or reversing the effects of hair thinning. The composition may include an effective amount of a hair loss preventative or hair growth promoting composition comprising a plant extract identified as Shea Butter in combination with papaya and polysaccharides.

### PRIORITY CLAIM

The present application claims priority from the following provisional patent application: U.S. provisional application Ser. No. 60/499,919, which was filed Sep. 4, 2003.

### I— BACKGROUND OF THE INVENTION

A. Field of the Invention

The present invention relates to a therapeutic preparation comprising an extract and the medical use thereof. Specifically, the present invention relates to a composition comprising the plant extract known as Shea Butter in combination with papaya and polysaccharides, having the effect of increasing or restoring hair growth, and/or preventing hair loss. More specifically, the present invention relates to a hair growth restoring and/or hair loss preventing composition containing the plant extract known as Shea Butter in combination with papaya and polysaccharides.

B. Description of Related Art

It is historically evident that humans have always taken care of their hair. Hair loss is a common problem in both men and women and it concerns over 42% of the male population in the world. The hair protects the scalp from trauma and ultraviolet-light, but it has also an important role in a sexual sense serving as a decoration. For this reason hair has been adored and worshipped in different populations, religious groups, mythologies, cultures, social classes and sciences. The loss of hair has an impact upon the sensitive psychological status of a human being. At all times and in all cultures baldness has been treated with various recipes, magical medicine extracts and exotic wonder medicines, but the results of different treatments have always been poor.

The recorded literature, including the medical, scientific and patent literature, relates various efforts to treat and/or prevent hair loss and to restore and/or encourage hair growth, particularly regarding hair on the human scalp. Some of these efforts have met with varying degrees of success, ranging from complete failure to more-recently available topically administered drugs such as Rogaine® (active ingredient, minoxidil) and orally administered drugs such as Propecia® (active ingredient, finasteride). The active ingredients in these two drugs have been approved by the United States Food and Drug Administration for promoting hair growth. Rogaine® and Propecia® exhibit some degree of success in promoting and/or restoring hair growth, particularly hair loss at the vertex or crown of the head, but administration of these drugs may lead to certain adverse side effects, including for example sexual dysfunction. Moreover, Rogaine® and Propecia® are to be used on a continual basis and are relatively expensive. Accordingly, individuals with thinning hair or hair loss, or individuals likely to experience thinning hair or hair loss, especially men with thinning hair or hair loss on the scalp or, more particularly, on the vertex of the head, are in need of alternative treatments to encourage and/or restore hair growth.

Accordingly, there have been a variety of efforts to fulfill this need. As evidence of these efforts, the United States Patent and Trademark Office (USPTO) has granted approximately 100 patents on methods and or compositions for treating hair loss and/or thinning hair. Among these patent are patents that disclose baldness remedies made from botanical, vegetative, or other found materials. For example, these patents include U.S. Pat. No. 5,679,378 (for the topical use of dead sea mud); U.S. Pat. No. 5,744,128 (for the topical use of emu oil); U.S. Pat. No. 5,665,342 (for the topical use of potato peelings and lantana leaves); U.S. Pat. No. 5,597,575 (for the topical use of vitamin D3 and aloe); U.S. Pat. No. 5,674,510 (for the topical use of garlic powder, brewers yeast, grapefruit, acidic acid and kelp); U.S. Pat. No. 5,750,108 (for the topical use of tea tree oil, chlorine dioxide and acidic solution and saw palmetto berry extract); U.S. Pat. No. 5,695,748 (for the topical use of sage, aloe and nettles, castor oil, shea butter, wheat germ oil and white iodine) and U.S. Pat. No. 5,494,667 (for the topical use of pine extract and bamboo extract or Japanese apricot). The United States Food and Drug Administration had not determined whether these methods and/or compositions of treating hair loss and restoring hair growth are uniformly safe and effective.

Shea Butter, commonly known as Karité, is derived from the Shea Nut Tree (butyrospermum parkii) which grows in the western region of Africa. The fruits of these trees contain a nut which is dried and ground. The powder is then boiled in water to release an unctuous substance which rises to the top and solidifies to create Shea Butter (Diop). This butter has been used for centuries in Africa to moisturize and protect the skin from sun, wind, heat and salt water.

Shea Butter can be used for all forms of massage as it creates a frictionless surface allowing for the smoothest and most therapeutic deep tissue work. It is an excellent base for the addition of essential oils. And it is a key ingredient in the most reputable French beauty products and soaps, only recently gaining recognition in the United States. Due to its unsaponifiable nature, Shea Butter does not rob the skin of its natural oils and can actually help stimulate collagen production. As such, it can be very effective in helping to alleviate wrinkles, scars and burns.

Shea Butter can be used as a hair dressing to moisturize a dry scalp and stimulate hair growth. Used as a pomade, it helps to hold the hairstyle and lightly relax curls.

Medicinally, Shea Butter has been studied as an anti-inflammatory topical cream, being helpful in cases of arthritis (Kerharo), and it contains stigmasterol which is the sterol known as “the anti-stiffness factor” making it helpful in cases of rheumatism (Hampton). It has additionally been studied as a nasal decongestant by applying it to the inside of the nostrils (Tella). Shea Butter is also helpful in cases of eczema and dermatitis and “contains chemical constituents that help to heal bruising and soreness” (Falconi).

Shea Butter contains a high content of cinnamic acid creating a mild natural sunscreen, approximately SPF-6 (Falconi) and can be used alone to prevent burning (Hampton).

However, the use of Shea Butter alone or in combination with papaya and polysaccharides to prevent hair loss or to facilitate hair growth is not known. The present invention makes it possible to prevent hair loss or to facilitate hair growth for the first time in a satisfactory manner.

### II—SUMMARY OF THE INVENTION

The present invention is based on the principle that certain plant extracts can enhance hair growth and hair restoration for damaged hair. Plant extracts can be administered topically to enhance hair growth and hair restoration for damaged hair.

The present invention includes methods for the treatment and/or prevention of hair loss and methods for the regeneration or restoration of hair growth comprising a step of identifying an individual suffering from or susceptible to hair loss or hair thinning or in need of hair regeneration, and a step of administering an extract of known as Shea Butter in combination with papaya and polysaccharides. Preferably, the extract is an aqueous extract and the composition containing the Shea butter is administered topically. The composition is most preferably comprised of the plant extract Shea Butter in combination with papaya and polysaccharides. The present invention also provides a composition, preferably in the from of a lotion, gel, cream, or other suspension, effective in restoring hair growth, preventing hair loss, and/or reversing the effects of hair thinning. The composition might contain an additional distinct chemical compound or class of chemical compounds therein. The composition may include an effective amount of a hair loss preventative or hair growth promoting composition comprising an extract known as Shea Butter, papaya and polysaccharides.

These and other objects, features and advantages of the invention will be clear to a person of ordinary skill in the art upon reading this specification.

### III. DETAILED DESCRIPTION OF A PREFERRED EMBODIMENT

A. Composition of the Invention

One object of the present invention is to provide a non-toxic hair growth restorer and/or hair loss preventer. Another object of the present invention is to provide a relatively inexpensive hair growth restoring composition or hair loss preventing composition, including a composition comprising the Shea Butter, papaya and polysaccharides having the effect of restoring hair growth and/or preventing hair loss. Another objective of the present invention is to provide an a topically-applicable composition for restoring hair growth and/or preventing hair loss. Yet another object of the present invention is to provide a renewable resource for a hair growth restorer or hair loss preventing a composition comprising the Shea Butter, papaya and polysaccharides. The preferred hair loss preventing and/or hair growth restoring composition of the present invention is a composition comprising the Shea Butter, papaya and polysaccharides. The more preferred hair loss preventing and/or hair growth restoring composition of the present invention is a composition comprising about ⅓ Shea Butter, about ⅓ papaya, and about ⅓ polysaccharides.

Shea Butter can be used for all forms of massage as it creates a frictionless surface allowing for the smoothest and most therapeutic deep tissue work. It is an excellent base for the addition of essential oils. And it is a key ingredient in the most reputable French beauty products and soaps, only recently gaining recognition in the United States. Due to its unsaponifiable nature, Shea Butter does not rob the skin of its natural oils and can actually help stimulate collagen production. As such, it can be very effective in helping to alleviate wrinkles, scars and burns.

Shea Butter can be used as a hair dressing to moisturize a dry scalp and stimulate hair growth. Used as a pomade, it helps to hold the hairstyle and lightly relax curls.

Medicinally, Shea Butter has been studied as an anti-inflammatory topical cream, being helpful in cases of arthritis (Kerharo), and it contains stigmasterol which is the sterol known as “the anti-stiffness factor” making it helpful in cases of rheumatism (Hampton). It has additionally been studied as a nasal decongestant by applying it to the inside of the nostrils (Tella). Shea Butter is also helpful in cases of eczema and dermatitis and “contains chemical constituents that help to heal bruising and soreness” (Falconi).

Shea Butter contains a high content of cinnamic acid creating a mild natural sunscreen, approximately SPF-6 (Falconi) and can be used alone to prevent burning (Hampton).

The polysaccharides can be chosen from natural and modified polysaccharides. Natural polysaccharides include starches, e.g., starches from vegetable origins such as maize, potato, oats, rice, tapioca, sorghum, barley and wheat; polysaccharides from seed gums such as guar, tara, carob, psyllium, linseed, okra, tamarind, quince gums; polysaccharides from microbial gums such as curdlan, pullulan, dextran, grifolan, schizophyllan, spirulinan, krestin, xanthan, scleroglucan, gellan, succinoglycan gums; polyfructoses such as inulin and levan; algae extracts (agar, alginate, caragheenans, fucoidane, furcellerane, laminarane); extracts from plant exudates (gum arabic, ghatti gum, karaya gum, adraganth gum); celluloses; and chitins.

Modified polysaccharides means polymers obtained by hydrolysis, esterification, etherification, amidation, oxidation, reduction, modification of the hydroxyl groups, covalent incorporation of organometallic residues, grafting, N-acylation, N-alkylation, deamination, or any other modification of the nitrogen-containing functions of polysaccharides containing said functions and/or halogenation using processes known in the art.

Examples of appropriate processes include:

(a) partial hydrolysis of polysaccharide chains using enzymatic treatment, heat treatment, acid treatment or oxidizing treatment;
(b) partial or complete hydrolysis of naturally present ester functions;
(c) partial or complete esterification and/or amidation of naturally present carboxylic functions;
(d) carboxylation using, for example, monochloroacetate;
(e) oxidation of hydroxyl functions in the polysaccharide;
(f) polyoxyalkylenation;
(g) hydrophobic modification of a polysaccharide, for example by reacting a C₁-C22 alkylating agent such as methyl chloride or nonyl chloride on alkali cellulose or on hydroxyethyl cellulose;
(h) crosslinking of polysaccharide chains; and
(i) grafting, for example, resulting in introducing into the molecule moieties such as polydimethylsiloxane, polyacrylic acid or polymethacrylic acid, polyacrylamide, polymethacrylamide, polyacrylonitrile, sodium polystyrene sulphonate, polyvinylpyrrolidone, polyhydroxyalkyl(meth)acrylate, or the sodium salt of poly(2-acrylamido-2-methylpropanesulphonic) acid.
   Modified polysaccharides include hydroxyalkyl celluloses, such as hydroxyethyl celluloses, hydroxyalkyl guars such as hydroxypropyl guar, carboxyalkyl celluloses, carboxyalkyl starches, modified and unmodified chitosans, maltodextrins and cyclodextrins.

As used herein, the terms “restore hair growth,” “hair restorer,” and “to restore hair growth” are essentially interchangeable. Each refers to methods or compositions for increasing the amount of hair growth. These phrases do not necessarily refer to the production of a full head of hair, nor do they refer to the restoration of hair growth to the state before the onset of hair loss or hair thinning. Most typically, although not exclusively, “restore hair growth” refers to increasing the amount of hair growth at the vertex or crown of the head or at the front of the hair near the hair line, and most typically, although not exclusively, refers to human males.

As used herein, the terms “prevent hair loss,” “hair loss preventer,” and “to prevent hair loss” are essentially interchangeable. Each refers to methods or compositions for preventing the degree of hair loss. “Hair loss,” as that term is used herein, includes hair thinning. These phrases do not necessarily refer to the complete cessation of hair loss or of hair thinning; rather, they refer to any measurable slowing in the rate of hair loss, as measured by standard measurements such as follicle count per unit skin area or by mass of hair per unit skin area. Such hair loss may be brought on by any of a variety of conditions, as will be understood by those of skill in the art. Most typically, although not exclusively, “prevent hair loss” refers to preventing the amount of hair lost at the vertex or crown of the head or at the front of the hair near the hair line, and most typically, although not exclusively, refers to human males.

As used herein, the phrases “hair loss” and “thinning hair,” both refer to the state in which fewer hair follicles per unit area (hair count) on the skin are produced than in a state prior to the onset of hair loss or hair thinning, and may also refer to the state in which less mass of hair per unit skin area is produced than in a state prior to the onset of hair loss or hair thinning. Most typically, although not exclusively, “hair loss” refers to the amount of hair at the vertex or crown of the head or at the front of the hair near the hair line, and most typically, although not exclusively, refers to human males.

Preferred methods of the present invention generally include a first step of identifying an individual suffering from hair loss or hair thinning, or likely to experience hair loss or hair thinning. One such condition, which may be linked to a genetic marker. Identification of such a condition, or the likelihood of experiencing such a condition, may be made by direct observation, as appreciated by those of skill in the art, or by evaluation of the phenotype of an individual's genetic relatives. As will be understood by those of skill in the art, an individual's likelihood of experiencing hair loss generally increases as that individual's genetic relatives, most typically the male, maternal genetic relatives of the individual, are identified as having experienced hair loss or hair thinning. This observation is especially true of human males. Furthermore, genetic markers are identifiable for identifying individuals likely to experience hair loss of hair thinning; these markers may be used to identify an individual or the individual's genetic relatives consistent with the first step of the preferred methods of the present invention. Most preferably, an individual is identified by direct observation of hair loss or hair thinning over time. Such direct observation may be by the individual, a member of the individual's family, or by another, including but not limited to hair-care or medical professionals.

Preferred methods of the present invention generally include a subsequent step of applying or administering a composition comprising an extract known as Shea Butter, papaya and polysaccharides. The extract may be either an aqueous extract, an alcoholic extract, or an organic extract. Most preferably, the extract is an aqueous extract.

The preferred hair loss preventing and/or hair growth restoring composition of the present invention is a simple plant composition comprising the Shea Butter, papaya and polysaccharides. The more preferred hair loss preventing and/or hair growth restoring composition of the present invention is a composition comprising about ⅓ Shea Butter, about ⅓ papaya, and about ⅓ polysaccharides.

In other embodiments, this composition may include, but need not necessarily include, pharmaceutically acceptable carriers that will allow the composition to be prepared for storage and subsequent administration. Such compositions, nonetheless, should contain a pharmaceutically effective amount of the compounds or class of compounds of the present invention, in a pharmaceutically acceptable carrier or diluent. Such acceptable carriers or diluents are described, for example, in Remington's Pharmaceutical Sciences, Mack Publishing Co. (A. R. Gennaro edit. 1985). Preservatives, stabilizers, dyes, or aromatic agents (especially where the composition is topically applied) may be provided in the pharmaceutical composition. Also, sodium benzoate, sorbic acid and p-hydroxybenzoic acid esters, antioxidants, and/or suspending agents may be added as preservatives.

The safety and efficacy of the preferred compound of class of compounds effective in preventing hair loss and/or restoring hair growth may be established by any of a variety of standard such as animal models or human clinical trials. When choosing an appropriate model to determine efficacy of a method of composition or compound, or class of compound of the present invention, the skilled artisan will be capable of choosing an appropriate model, dose, and route of administration, and regime.

As will be appreciated by those of skill in the art, the preferred plant extract of this invention effective in preventing hair loss and/or restoring hair growth may be administered via a variety of methods, including but not limited to topical administration. The preferred method of administration is via topical administration directly the skin at the area effected, or likely to be effected by hair loss and/or hair thinning. Compositions for topical administration of for use can be prepared in conventional forms, either as liquid solutions or suspensions, solid forms suitable for solution or suspension in liquid prior to application, or as emulsions. Any of the known transdermal carriers can further be incorporated into topical formulations, such as DMSO or azone. Such transdermal carriers, and others, are known to facilitate delivery of topically-applied active ingredients across the stratum corneum (SC) and/or the stratum germinativum (SG). Accordingly, a topically applied composition having the hair loss preventing or hair growth restoring plant extract of the present invention may be a lotion, gel, or cream, using pharmaceutically-acceptable carriers, stabilizers, and excipients known to those of skill in the art. Suitable excipients, are, for example, water, saline, dextrose, mannitol, lactose, lecithin, albumin, sodium glutamate, cysteine hydrochloride, and the like. In addition, if desired, the topical of compositions may contain nontoxic auxiliary substances, such as wetting or buffering agents. In practicing the method of the invention, the compositions can be used alone or in combination with one another, or in combination with other therapeutic or diagnostic agents.

As will be appreciated by those of skill in the art, the preferred plant extract of this invention effective in preventing hair loss and/or restoring hair growth may be administered topically as a lotion, gel, cream, or other salves or suspensions. Such lotions, gels, creams, or salves may preferably include agents suitable for cleansing the skin and/or hair in the affected, treated area. Such agents include, but are not limited to, soaps, fatty acids, conditioners, and essential oils. Also, suitable thickeners, coloring agents, perfumes, aromatic agents, and/or preservatives may be added to the lotions, gels, creams, or other suspensions or salves as desired by the treated individual or the individual's companions. Such agents may be useful in disguising or enhancing the color and odor of the effective plant extract of this invention.

The composition of the present invention may be marketed and/or used in conjunction with shampoos, conditions, styling gels, or other hair care products. For example, the composition of the present invention may be marketed and/or used in conjunction with a shampoo and/or a conditioner that improves the appearance or apparent thickness of hair. The present invention includes, but is not limited to, a method of cosmetic treatment. More specifically, the present invention includes the use of the composition comprising Shea Butter, papaya and polysaccharides in the treatment and/or the prevention of hair loss, and/or in the restoration or hair growth. The present invention also includes, but is not limited to, the use of the composition comprising Shea Butter, papaya and polysaccharides for the preparation of a medicament useful in the treatment and/or the prevention of hair loss, and/or in the restoration or hair growth. Preferably, the present invention encompasses the use of the composition comprising Shea Butter, papaya and polysaccharides for the preparation of a medicament for the treatment and/or the prevention of hair loss, and/or in the restoration or hair growth, in a male or female human.

B. Extraction and Purification

Generally, Shea Butter, papaya, and polysaccharides can be extracted from plant material using various techniques that are known to those skilled in the art.

The present invention is described in detail through a variety of examples. It will be understood by those skilled in the art that the invention is not limited to the specific examples provided herein. Furthermore, although specific amounts of plant material and modes of extraction are specified in the following examples, it will be understood by those skilled in the art that the invention is not limited to these specific amounts and modes, and that variations in the amount of plant material, the mode or duration of extraction, and the method of administration, or more specifically, of application to the scalp and or skin, may be varied and still to achieve the desired effect of increasing the amount of hair growth and/or preventing hair loss or hair thinning.

### DRAWING DESCRIPTIONS

No drawings are included in this application.

Description:

Preparation of an Extract

Table 1 provides the active substances, and the proportions of these active substances that comprise the preparation. The following preparation is not intended to be limiting, but are merely representative of the invention of the application
about ⅓ Shea Butter about ⅓ papaya about ⅓ polysaccharides

While the present invention has been described and illustrated in conjunction with a specific embodiment, those skilled in the art will appreciate that variations and modifications may be made without departing from the principles of the invention as herein illustrated and described.

The invention may be embodied in other specific forms without departing from the spirit or essential characteristics thereof. The present embodiments are to be considered in all respects as illustrative, and not restrictive.

### LIST OF REFERENCES:

Essential oil information from Aromatherapy for Mother and Child by Jane Dye, C.W. Daniel Company, England, 1992.
Falconi, Dina, Earthly Bodies and Heavenly Hair, Ceres Press, New York, 1998, 196.
Hampton, Aubrey, Natural Organic Hair and Skin Care, Organica Press, Florida, 1997, 147-48.
Kerharo, J., “Note sur les excipients pour pommades”, Centre de documentation pharmaceutique et chimique des troupes coloniales, Marseille, 1942.
Tella, A., “Preliminary studies on nasal decongestant activity from the seed of the shea butter tree Butyrosvpermum parkii”, Br. J. Clin. Pharmac., 1979, 7, 495-7.
Diop, Taïb. Les Plantes Medicinales, Dakar, Sénégal, 1996.
Tisserand, Maggie and Junemann, Monika, The Magic and Power of Lavender, Lotus Light, Wisconsin, 1994.

1. A composition for treatment or prevention of hair loss comprising: an effective amount of a hair loss preventative or hair growth promoting composition comprising Shea Butter, papaya, and polysaccharides.

2. The composition of claim 1, wherein the composition comprises about ⅓ Shea Butter, about ⅓ papaya, and about ⅓ polysaccharides

3. The composition of claim 1, wherein the extract is an aqueous extract.

4. The composition of claim 1, wherein the extract is applied with a carrier.

5. The composition of claim 1, wherein the extract is applied with an aqueous carrier.

6. The composition of claim 1, wherein the carrier is a non-aqueous carrier.

7. The composition of claim 1, wherein the extract is partially isolated from plants.

8. The composition of claim 1, wherein the extract is partially chemically synthesized.

9. The composition of claim 1, wherein the composition is selected from a lotion, a gel, and a cream.

10. A composition for treatment or prevention of hair loss comprising: an effective amount of a hair loss preventative or hair growth promoting composition wherein said composition consists of Shea Butter, papaya, and polysaccharides.

11. The composition of claim 10, wherein the composition consists of about ⅓ Shea Butter, about ⅓ papaya, and about ⅓ polysaccharides

12. The composition of claim 10, wherein the extract is an aqueous extract.

13. The composition of claim 10, wherein the extract is applied with a carrier.

14. The composition of claim 10, wherein the extract is applied with an aqueous carrier.

15. The composition of claim 10, wherein the carrier is a non-aqueous carrier.

16. The composition of claim 10, wherein the extract is partially isolated from plants.

17. The composition of claim 10, wherein the extract is partially chemically synthesized.

18. The composition of claim 10, wherein the composition is selected from a lotion, a gel, and a cream.

19. A process for conditioning damaged, thinning hair and for restoring hair growth which comprises the steps of:
applying a composition comprising Shea Butter, papaya, and polysaccharides to the hair and scalp in an amount and for a period of time effective to treat the hair and scalp.

20. The process of claim 19 wherein the quantities, by weight, of Shea Butter, papaya, and polysaccharides employed differ from each other.

21. The process of claim 19 wherein the quantities, by weight, of Shea Butter, papaya, and polysaccharides employed are the same.

22. A method for restoring hair growth or preventing thinning hair comprising the steps of:
identifying an individual suffering from or likely to suffer from hair loss or thinning hair; and administering an effective amount of a composition comprising Shea Butter, papaya, and polysaccharides

23. The method of claim 22, wherein the extract comprising about ⅓ Shea Butter, about ⅓ papaya, and about ⅓ polysaccharides

24. The method of claim 22, wherein the extract is an aqueous extract.

25. The method of claim 22, wherein the individual is a human male.

26. The method of claim 22, wherein the extract is administered via topical administration to an area that has experienced or likely will experience hair loss or thinning hair.

27. The method of claim 26, wherein the area that has experienced or that likely will experience hair loss comprises a portion of the human scalp.

28. The method of claim 26, wherein the area has experienced hair loss.

29. Preparation for treating hair consisting of:
about ⅓ Shea Butter about ⅓ papaya about ⅓ polysaccharides

30. A topical reparation for treating hair consisting of:
Shea Butter
papaya
polysaccharides

31. A topical preparation for treating hair consisting of:
about ⅓ Shea Butter about ⅓ papaya about ⅓ polysaccharides
